(19) United States
(12) Patent Application Publication
(10) Pub. No.: US 2010/0074846 A1
(43) Pub. Date: Mar. 25, 2010

(54) Campylobacter Vaccines and Methods of use

(76) Inventors: John A. Ellis, Saskatoon (CA); George Steven Krakowka, Columbus, OH (US); Kathleen Anne McIntosh, Saskatoon (CA)

Correspondence Address: ROBINS & PASTERNAK, 1731 EMBARCADERO ROAD, SUITE 230, PALO ALTO, CA, 94303, US

(21) Appl. No.: 12/225,145

(22) PCT Filed: Mar. 2, 2007
(86) PCT No.: PCT/US2007/005135

§ 371 Date: Oct. 20, 2009

Related U.S. Application Data
(60) Provisional application No. 60/783,499, filed on Mar. 17, 2006.

(51) Int. Cl. A61K 49/00; A61K 39/02; A61P 31/04

(52) U.S. Cl. 424/9.2; 424/234.1

(57) ABSTRACT

Porcine models for studying bacterial gastritis and gastric and duodenal ulcer disease caused by Campylobacter pathogens, such as C. coli are described, as well as methods of identifying vaccines and compounds for treating and/or preventing Campylobacter infection using the animal models. Also described are methods of preventing Campylobacter infection in swine, such as infection caused by C. coli, using immunogenic proteins and nucleic acids derived from Campylobacter pathogens, such as C. coli.

### CROSS-REFERENCE TO RELATED APPLICATION

This application claims the benefit of U.S. Provisional Application Ser. No. 60/783,499 pursuant to 35 U.S.C. §119(e), which application is incorporated herein by reference in its entirety.

### TECHNICAL FIELD

The present invention relates generally to bacterial pathogens. In particular, the invention pertains to vaccines for use in methods of treating and preventing Campylobacter infection in swine, as well as reducing bacterial load in swine to limit food-borne transmission of zoonotic pathogens. The invention also relates to animal models for studying bacterial gastritis and gastric and duodenal ulcer disease caused by Campylobacter spp. such as C. coli and C. jejuni.

### BACKGROUND

Gastric disease is an important cause of morbidity and economic loss in swine-rearing operations (O'Brien, J. (1992) “Gastric ulcers” p. 680. In A. D. Leman, B. E. Straw, W. L. Mengeling, and S. D. D'Allaire (ed), Diseases of swine. Wolfe, London, United Kingdom). Although the cause of porcine gastric disease has not been previously established, it is most often attributed to diet and/or stress (O'Brien, J. (1992) “Gastric ulcers” p. 680. In A. D. Leman, B. E. Straw, W. L. Mengeling, and S. D. D'Allaire (ed), Diseases of swine. Wolfe, London, United Kingdom).

Of the domestic animal species, swine are the most commonly affected with clinically significant gastric ulcers (O'Brien J. J. Gastric Ulcers. Diseases of Swine, 6th ed. Editors A D Leman A D, et al., (1986), 680-691; Embaye et al. (1990) J. Comp. Path. 103:253-264). In modern swine-intensive production systems, the development of ulcers and erosions of the nonglandular esophageal (cardiac) gastric lining and antral gastric mucosa is a common and serious disease problem (O'Brien J. J. Gatric Ulcers. Diseases of Swine, 6th ed. Editors A D Leman A D, et al., (1986), 680-691). A prevalence of 5-100% for gastroesophageal ulcerations (GEU) is reported, death losses from fatal hemorrhages of 3% or more are reported (O'Brien. J. J. Gastric Ulcers. Diseases of Swine, 6th ed. Editors A D Leman A D, et al., (1986), 680-691; Embaye et al. (1990) J. Comp. Path. 103:253-264) and sublethal economic losses are substantial.

Porcine gastric mucosal ulceration and GEU are attributed to reflux of acidic gastric contents onto the unprotected pars esophagea (Argenzio et al. (1975) Am. J. Physiol. 228:454-462; Argenzio et al. (1996) Am. J. Vet. Res. 57:564-573). In particular, the stratified squamous epithelium of the pars esophagea is devoid of mucous-producing glands and lacks the sodium bicarbonate buffering system characteristic of the gastric glandular mucosa and, as a consequence, the pars is frequently damaged by the acidic contents of the stomach. Elevated gastric acid content is multifactorial and thought to be largely due to a combination of excess parietal cell production of hydrochloric acid, luminal hydrolysis of luminal carbohydrate, both coupled with a loss of pH gradient in the stomachs of swine fed a finely ground low roughage high carbohydrate diet.

As in humans with recurrent “heart burn,” reflux esophagitis and Barrett's esophagus, it is believed that gastric-origin hydrogen ions and acidic metabolites of partial intragastric glycolysis enter and acidify the squamous epithelial cell cytoplasm. The cell membrane-bound Na-K-ATPase is disrupted which results in accumulation of intracellular sodium ions and secondary accumulation of intracellular water, recognized histologically as acute cellular swelling, hydropic degeneration, epithelial parakeratosis and ultimately necrosis. For erosive lesions, the underlying basement membrane remains intact and re-epithelization of the damaged portion of the pars is rapid. Presumably pivotal to progression of epithelial erosions to ulceration is penetration of the basement membrane and continued acid-mediated damage to the underlying lamina propria. This devitalized tissue may be secondarily colonized by commensal microbes including fermentative anaerobes. In humans as well as swine, there is a strong consensus that a relative or absolute increase in gastric hydrogen ions (acid) is the proximate cause of pars and esophageal damage. Thus, a therapeutic goal in humans is to elevate gastric pH towards neutrality through the use of bicarbonate buffering medications and to inhibit new gastric hydrogen ion production by parietal cells of the gastric fundus with proton pump inhibitors. These over-the-counter medications provide immediate symptomatic relief for patients affected with heart burn and reflux esophagitis and indirectly implicate gastric hyperacidity in the pathogenesis of disease. However, such medications do not cure the underlying cause of the disease.

Feeder swine diets contain unsaturated fatty acids, short chain (acetate, propionate, butyrate and lactate) free fatty acids or peroxidized fats, all of which elevate luminal acid concentration (Argenzio et al. (1975) Am. J. Physiol. 228:454-462). Finishing diets high in carbohydrate such as corn and cornstarch are also a primary dietary source of acidic metabolites in pigs. Incomplete glycolysis of cornstarch by parietal cell-origin hydrogen ions and/or enzymatic actions of commensal fermentative microbes such as the Lactobacillus and Bacillus spp. results in the generation of lactic, acetic and propionic acids within the gastric compartment. Indeed it has been demonstrated that gastric colonization with fermentative bacterial species resulted in GEU if a dietary source of carbohydrate (corn syrup) was provided to colonized gnotobiotes (Krakowka et al. (1998) Vet. Pathol. 35:274-282). Finally, in feeder swine, the physical form of diet also influences development of GEU (O'Brien S. J. Gastric Ulcers. Diseases of Swine, 6th ed. Editors A D Leman A D, et al., (1986), 680-691). In general, a finely ground (<3.5 mesh) diet, even in pelleted form is an important risk factor for ulcerogenesis presumably because of the general inability of these diets to “confine” released acids to the fermentation compartment of the glandular stomach. The loss of a pH gradient associated with finely ground diets permits cranial acid reflux into the pars esophagea.

In 1984, Helicobacter pylori (Hp) emerged as an etiologic agent in human gastritis/ulcer disease following the documentation of this agent in patients with gastritis (Marshall and Warren (1984) Lancet 1:1311-1314). Hp is a Gram-negative microaerophilic urease-positive small curved rod-shaped bacterium which possesses several unusual characteristics related to its gastric ecological niche. The hallmark for the members of the Helicobacter genus is expression of urease enzyme. The presence of this enzyme and its activity in the hydrolysis of urea forms the basis of presumptive tests (urea breath test and others) for gastric colonization. The organism colonizes the mucus layer of the gastric cardia and antrum and infection is presumed to be lifelong.

Hp is now universally recognized as one of the primary gastric pathogens and the study of this bacterial species and the spectrum of diseases associated with it has become a major focus in human gastroenterology (Suerbaum and Michetti (2002) N. Eng. J. Med. 347:1175-1186). Hp is causally associated with chronic superficial (active) type B gastritis (Buck (1990) Clin. Micro. Rev. 3:1-12; Blaser (1992) Gasteroenterol. 102:720-727; Consensus Statement, 1994, NSAID), independent gastric ulceration (Peterson (1991) N. Eng. J. Med. 324:1043-1047; Moss and Calam (1992) Gut 33:289-292; Leung et al. (1992) Am. J. Clin. Pathol. 98:569 574; Forbes et al. (1994) Lancet 343:258-260), atrophic gastritis (Nomura et al. (1991) N. Engl. J. Med. 325:1132-1136; Parsonnet et al. (1991) JNCI 83:640-643; Sipponen (1992) Drugs 52:799-804, 1996), and gastric MALT lymphoma (Rodriguez et al. (1993) Acta Gastro-Enterol. Belg. 56 (suppl):47; Eidt et al. (1994) J. Clin. Pathol. 47:436-439). Additionally, atrophic gastritis and resultant acholrhydria is now thought to represent the last stage in the progression of persistent lifelong colonization by Hp (Leung et al. (1992) Am. J. Clin. Pathol. 98:569 574).

Multiple agent antimicrobial therapies have been available for human Hp for more than a decade. These therapies can be expensive, cumbersome to administer, and often do not completely cure the disease. Such therapies would be impractical in domestic livestock. Moreover, injudicious use of antimicrobials promotes emergence of antibiotic-resistant strains of Hp and Hp resistance to metronidazole and clarirythromycin has increased (Michetti, (1997) Gut 41:728-730). Additionally, the use of antibiotics in food animals is undesirable.

Recently, a new Helicobacter pathogen was recovered from swine exhibiting gastritis/ulcer disease. This pathogen, named H. cerdo, has been shown to cause gastric disease in young piglets that is similar to Hp-associated active gastritis in humans. H. cerdo is described in detail in PCT Publication No. WO 2004/069184.

Campylobacter spp have long been implicated as etiologic agents in enteritis in swine. Agents such as C. jejuni and C. coli are recognized as important zoonotic and food-borne pathogens that can cause enteric disease in humans. Animal models that mimic Campylobacter infection are of great use in studying treatment and prevention options. Of increasing concern is accumulating data indicating extant and expanding antibiotic resistance among Campylobacter species, including C. coli, which will make their control by traditional chemotherapeutic modalities increasingly difficult. This coupled with the increasing concern about antibiotic use in food-producing animals such as swine, make the development of vaccines to reduce pathogen load and disease caused by Campylobacter spp a necessity. C. coli is recognized as a highly prevalent bacterium in swine populations worldwide. This agent colonizes both the stomach and small intestines. Despite its common occurrence in the stomach of pigs, to date no investigations have attempted to link colonization of the stomach by C. coli with gastritis and GEU in swine.

### SUMMARY OF THE INVENTION

The present invention is based in part on the discovery that C. coli is an etiologic agent in gastritis and GEU in swine. In particular, the present inventors have isolated C. coli from the stomachs of naturally infected pigs with gastritis and GEU. These isolates were used to reinfect swine and C. coli was then reisolated from gastric lesions in the experimentally infected pigs, indicating that C. coli is an additional, previously unrecognized, etiologic agent in gastritis and GEU in swine.

Thus, Campylobacter immunogens can be used in porcine vaccines for gastritis and GEU. Such vaccines serve the dual purpose of protecting pigs from Campylobacter infection, as well as eliminating health hazards to the public by protecting humans from food-borne transmission of Campylobacter pathogens.

Campylobacter immunogens can also be used to create animal models that reproduce porcine Campylobacter infection. The animal models include gnotobiotic piglets or conventionally-reared pigs that are immunized with vaccine candidates and then challenged with Campylobacter bacteria, such as C. coli. In some embodiments, the piglets are inoculated with C. coli and optionally fed a milk-replacement diet containing a dietary source of fermentable carbohydrate in order to simulate porcine gastroesophageal ulceration (GEU). The animal models are useful for identifying compounds and compositions, such as C. coli vaccine candidates, that have the ability to prevent or treat Campylobacter infection in humans and animals, such as swine.

Accordingly, in one embodiment, the invention is directed to a method of treating or preventing Campylobacter infection in a porcine subject comprising administering to the subject a therapeutically effective amount of a composition comprising at least one Campylobacter immunogen. In certain embodiments, the immunogen is a C. coli immunogen. In yet further embodiments, the composition comprises a C. coli lysate, such as a lysate produced by proteolytic digestion of C. coli bacteria. In further embodiments, the composition further comprises an adjuvant. In yet additional embodiments, the composition is administered orally.

In other embodiments, the invention is directed to a method for infecting a gnotobiotic piglet with a porcine isolate of Campylobacter. The method comprises:

(a) isolating Campylobacter from a porcine subject; and

(b) administering a dose of the Campylobacter isolate to the gnotobiotic piglet in an amount sufficient to cause Campylobacter infection.

In certain embodiments, the Campylobacter isolate is C. coli. In additional embodiments, the Campylobacter isolate is administered orally to the piglet. In some embodiments, the Campylobacter is administered in an amount of 10⁷-10⁹ colony forming units.

In yet further embodiments, the invention is directed to a method for evaluating the ability of a vaccine to prevent Campylobacter infection. The method comprises:

(a) administering to a gnotobiotic piglet a candidate vaccine;

(b) exposing the gnotobiotic piglet from step (a) to a Campylobacter isolate in an amount sufficient to cause infection in an unvaccinated subject; and

(c) observing the incidence of Campylobacter infection in the gnotobiotic pig, thereby evaluating the ability of the candidate vaccine to prevent Campylobacter infection.

In certain embodiments, the candidate vaccine is a C. coli vaccine comprising at least one C. coli immunogen and the Campylobacter isolate is a C. coli isolate.

In additional embodiments, the invention is directed to a method of producing a porcine animal model of gastroesophageal ulceration (GEU) of the pars esophagea. The method comprises:

(a) isolating Campylobacter from a porcine subject;

(b) exposing a gnotobiotic piglet to the Campylobacter isolate in an amount sufficient to cause infection in the piglet; and

(c) feeding the infected piglet a milk-replacement diet that contains a dietary source of fermentable carbohydrate under conditions sufficient for producing GEU of the pars esophagea.

In certain embodiments, the Campylobacter isolate is a C. coli isolate. In further embodiments, the Campylobacter isolate is administered orally to the piglet. In yet additional embodiments, the Campylobacter isolate is administered in an amount of 10⁷-10⁹ colony forming units. In other embodiments, the dietary source of fermentable carbohydrate is corn syrup.

In yet a further embodiment, the invention is directed to a method of producing a porcine animal model of gastroesophageal ulceration (GEU) of the pars esophagea. The method comprises:

(a) isolating C. coli from a porcine subject;

(b) orally administering 10⁷-10⁹ colony forming units of the C. coli isolate to a gnotobiotic piglet in order to cause C. coli infection; and

(c) feeding the infected piglet a milk-replacement diet that contains corn syrup as a fermentable source of carbohydrate under conditions sufficient for producing GEU of the pars esophagea.

In an additional embodiment, the invention is directed to a method of identifying a compound capable of treating Campylobacter infection. The method comprises:

(a) exposing a gnotobiotic piglet to a Campylobacter isolate in an amount sufficient to cause infection in the piglet;

(b) delivering a compound or series of compounds to the infected piglet; and

(c) examining the piglet from step (b) for the presence or loss of Campylobacter bacteria and/or the development, inhibition, or amelioration of ulcer or tumor formation relative to an untreated Campylobacter-infected gnotobiotic piglet.

In additional embodiments, the invention is directed to a method of identifying a compound capable of treating C. coli infection. The method comprises:

(a) providing a porcine animal model of GEU produced by any one of the Methods described above;

(b) delivering a compound or series of compounds to the infected piglet; and

(c) examining the piglet from step (b) for the presence or loss of C. coli bacteria and/or the development, inhibition, or amelioration of ulcer or tumor formation relative to an untreated C. coli-infected gnotobiotic piglet.

These and other embodiments of the subject invention will readily occur to those of skill in the art in view of the disclosure herein.

### DETAILED DESCRIPTION OF THE INVENTION

The practice of the present invention will employ, unless otherwise indicated, conventional techniques of molecular biology, microbiology, bacteriology, recombinant DNA technology, and immunology, which are within the skill of the art. Such techniques are explained fully in the literature. See, e.g., Sambrook, Fritsch & Maniatis, Molecular Cloning: A Laboratory Manual, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

All publications, patents and patent applications cited herein, whether supra or infra, are hereby incorporated by reference in their entirety.

### 1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a C. coli immunogen” includes a mixture of two or more such immunogens, and the like.

By “Campylobacter infection” is meant any disorder caused by a Campylobacter bacterium, including without limitation, C. coli, C. jejuni, C. lari, C. upsaliensis and C. hyotntestinalis, such as, but not limited to, chronic superficial (active) type B gastritis, independent gastric ulceration, peptic, gastric and duodenal ulcers, gastroesophageal ulceration (GEU), proventricular ulcers, ulcerative gastric hemorrhage, atrophic gastritis, and carcinoma including Campylobacter-associated immunoproliferative small intestinal disease (IPSID), a form of lymphoma that arises in the small intestinal mucosa-associated lymphoid tissue (MALT). See, e.g., Lecuit et al., New. Eng. J. Med (2004) 350:239-248. The term also intends subclinical disease, e.g., where Campylobacter infection is present but clinical symptoms of disease have not yet manifested themselves. Subjects with subclinical disease can be asymptomatic but are nonetheless at a considerable risk of developing peptic ulcers and/or gastric adenocarcinomas. For a review of Camplyobacter-associated diseases, see, Campylobacter, 2nd Edition (2000) Eds. Nachamkin and Blaser.

By “a Campylobacter lysate” is meant an extract or lysate derived from a Campylobacter whole bacterium, such as a C. coli whole bacterium, which includes one or more Campylobacter immunogens or immunogenic polypeptides, as defined below. The term therefore is intended to encompass crude extracts that contain several Campylobacter immunogens as well as relatively purified compositions derived from such crude lysates which include only one or few such immunogens. Such lysates are prepared using techniques well known in the art, described further below.

Representative immunogens that may be present in such lysates, either alone or in combination, include immunogens with one or more epitopes derived from any of the various Campylobacter flagellins, including but not limited to C. coli flagellins, such as FlaA and/or FlaB. Flagellin has been shown to be an immunodominant protein (Martin et al, Infect. Immun. (1989) 57:2542-2546; Wenman et al., J. Clin. Microbial. (1985) 21:108-112). The major immune response to flagellin appears to reside in the highly conserved amino and carboxy terminal ends of the protein, areas that are not surface-exposed in the flagellar filament. However, a truncated mutant in which a highly conserved region of FlaA fused to an E. coli maltose binding protein has been shown to be immunogenic.

Other Campylobacter virulence factors and toxins may also be present, such as but not limited to any of the various Campylobacter cytotoxins. Several such cytotoxins have been identified. See, e.g., Johnson and Lior, Microbiology pathogens (1988) 4:115-126; McFaland and Neill, Vet. Microbial. (1992) 30:257-266; and Schulze et al., Zentralblatt fur Bakteriologie (1998) 288:225-236. One particular cytotoxin is the cytolethal distending toxin (CDT) (Pickett et al., Infect. Immun. (1996) 64:2070-2078), encoded by the cdt gene cluster, including cdtA, cdtB and cdtC. Other virulence factors that can be present include phospholipase A(2) (Istivan et al., J. Med. Microbial. (2004) 53:483-493); Campylobacter enterotoxins (Lindblom et al., J. Clin. Microbial. (1989) 27:1272-1276); adhesins, such as CadF (Konkel et al., J. Clin. Microbial. (1999) 37:510-517; Konkel et al., Molec. Microbial. (1997) 24:953-963); and the lipoprotein encoded by ceuE which is a component of a protein-binding-dependent transport system for the siderophore enterochelin (Park and Richardson, J. Bacteria (1995) 177:2259-2264; Gonzalez et al., J. Clin. Microbial. (1997) 35:759-763); and the plasmid pVir (Bacon et al., Infect. Immun. (2000) 68:4384-4390).

Lysates may also contain PEB1 which plays an important role in endothelial cell interactions and colonization in mice (Pei and B laser, J. Biol. Chem. (1991) 59:2259-2264; Pei et al., Infect. Immun. (1998) 66:938-943). Pilin subunit proteins may also be present.

Campylobacter glycolipids, such as the lipo-oligosaccharide (LOS) and/or capsular polysaccharide (CPS) may also be present. See, e.g., Karlyshev et al., in Campylobacter: molecular and cellular biology (2005) Eds. Ketley and Konkel, Chapter 12.

See, e.g., Campylobacter, 2nd Edition (2000) Eds. Nachamkin and Blaser; Bang et al., J. Appl. Microbial. (2003) 94:1003-1014; Campylobacter: molecular and cellular biology (2005) Eds. Ketley and Konkel, for a description of these and other Campylobacter immunogens that can be present in such lysates.

It is to be understood that the lysate can also include other immunogens not specifically described herein.

By “subunit vaccine composition” is meant a composition containing at least one immunogen, but not all antigens, derived from or homologous to an antigen from the Campylobacter pathogen of interest. Such a composition is substantially free of intact pathogen cells or particles, or the lysate of such cells or particles. Thus, a “subunit vaccine composition” is prepared from at least partially purified (preferably substantially purified) immunogens from the Campylobacter pathogen, or recombinant analogs thereof. A subunit vaccine composition can comprise the subunit antigen or antigens of interest substantially free of other antigens or polypeptides from the pathogen. Representative immunogens are described above.

By “mucosal” delivery is meant delivery of an antigen to a mucosal surface, including nasal, pulmonary, vaginal, rectal, urethral, and sublingual or buccal delivery.

The term “polypeptide” when used with reference to a Campylobacter immunogen, such as FlaA, refers to an immunogen such as FlaA, whether native, recombinant or synthetic, which is derived from any Campylobacter strain. The polypeptide need not include the full-length amino acid sequence of the reference molecule but can include only so much of the molecule as necessary in order for the polypeptide to retain immunogenicity and/or the ability to treat or prevent Campylobacter infection, such as C. coli infection, as described below. Thus, only one or few epitopes of the reference molecule need be present. Furthermore, the polypeptide may comprise a fusion protein between the full-length reference molecule or a fragment of the reference molecule, and another protein that does not disrupt the reactivity of the Campylobacter polypeptide. It is readily apparent that the polypeptide may therefore comprise the full-length sequence, fragments, truncated and partial sequences, as well as analogs and precursor forms of the reference molecule. The term also intends deletions, additions and substitutions to the reference sequence, so long as the polypeptide retains immunogenicity.

Thus, the full-length proteins and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the biological activity, are contemplated for use herein.

The term “analog” refers to biologically active derivatives of the reference molecule, or fragments of such derivatives, that retain activity, as described above. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

A “purified” protein or polypeptide is a protein which is recombinantly or synthetically produced, or isolated from its natural host, such that the amount of protein present in a composition is substantially higher than that present in a crude preparation. In general, a purified protein will be at least about 50% homogeneous and more preferably at least about 80% to 90% homogeneous.

By “biologically active” is meant a Campylobacter protein that elicits an immunological response, as defined below.

By “epitope” is meant a site on an antigen to which specific B cells and T cells respond. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or γδ T cells, directed specifically to an antigen or antigens included in the composition or vaccine of interest. Preferably, the host will display a protective immunological response to the Campylobacter immunogen(s) in question, e.g., the host will be protected from subsequent infection by C. coli and such protection will be demonstrated by either a reduction or lack of symptoms normally displayed by an infected host or a quicker recovery time.

The terms “immunogenic” protein or polypeptide refer to an amino acid sequence which elicits an immunological response as described above. An “immunogenic” protein or polypeptide, as used herein, includes the full-length sequence of the particular Campylobacter immunogen in question, including any precursor and mature forms, analogs thereof, or immunogenic fragments thereof. By “immunogenic fragment” is meant a fragment of the Campylobacter immunogen in question which includes one or more epitopes and thus elicits the immunological response described above.

Immunogenic fragments, for purposes of the present invention, will usually be at least about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the Campylobacter immunogen in question.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+MR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “effective amount” or “therapeutically effective amount” of a composition or agent, as provided herein, refer to a nontoxic but sufficient amount of the composition or agent to provide the desired “therapeutic effect,” such as to elicit an immune response as described above, preferably preventing, reducing or reversing symptoms associated with the Campylobacter infection. This effect can be to alter a component of a disease (or disorder) toward a desired outcome or endpoint, such that a subject's disease or disorder shows improvement, often reflected by the amelioration of a sign or symptom relating to the disease or disorder. For example, a representative therapeutic effect can render the subject negative for Campylobacter infection when gastric mucosa is cultured for a Campylobacter pathogen. Similarly, biopsies indicating lowered IgG, IgM and IgA antibody production directed against the Campylobacter pathogen are an indication of a therapeutic effect. Similarly, decreased serum antibodies against the Campylobacter pathogen are indicative of a therapeutic effect. Reduced gastric inflammation is also indicative of a therapeutic effect. The exact amount required will vary from subject to subject, depending on the species, age, and general condition of the subject, the severity of the condition being treated, and the particular components of the composition administered, mode of administration, and the like. An appropriate “effective” amount in any individual case may be determined by one of ordinary skill in the art using routine experimentation.

“Treatment” or “treating” Campylobacter infection includes: (1) preventing the Campylobacter disease, or (2) causing disorders related to Campylobacter infection to develop or to occur at lower rates in a subject that may be exposed to Campylobacter, such as C. coli, (3) reducing the amount of Campylobacter present in a subject, and/or reducing the symptoms associated with Campylobacter infection.

### 2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the finding that C. coli causes gastritis in pigs. Thus, new animal models useful for studying the pathogenesis, treatment and prevention of Campylobacter infection, such as C. coli infection, in pigs can be developed.

Gnotobiotic piglets can be used to study the ability of various Campylobacter vaccines, such as C. coli and C. jejeuni vaccines, to prevent Campylobacter infection. Additionally, gnotobiotic piglets infected with C. coli can be used to screen various compounds for their ability to treat Campylobacter infection caused by, e.g., C. coli or C. jejeuni.

Campylobacter immunogens can be used in porcine vaccines for gastritis and GEU. Such vaccines serve the dual purpose of protecting pigs from Campylobacter infection, as well as eliminating health hazards to the public by protecting humans from food-borne transmission of Campylobacter pathogens.

In order to further an understanding of the invention, a more detailed discussion is provided below regarding the Campylobacter animal models, Campylobacter vaccines, as well as various uses thereof.

Campylobacter Animal Models

The gnotobiotic piglet is especially suited for studying bacterial gastritis/ulcer disease. See, e.g., Krakowka et al. (1987) Infect. Immun. 55:2789-2796. Gnotobiotic swine are monogastric omnivores with gastric anatomy and physiology that closely replicates humans. This animal model may also be suited for studying Campylobacter infection and therefore for identifying vaccine candidates, such as C. coli vaccines (i.e., vaccines including one or more immunogens derived from C. coli), useful for preventing Campylobacter infection in pigs. Thus, a preferred use for the animal models of the invention is the development of vaccines for use in the prevention and/or treatment of Campylobacter infection in pigs and diseases associated therewith.

In this context, pigs are administered the vaccine candidate at least once, and preferably boosted with at least one additional immunization. For example, gnotobiotic piglets can be administered a vaccine composition to be tested at 1-5 days of age, followed by a subsequent boost 5-10 days later, and optionally a third immunization 5-10 days following the second administration. Piglets can be vaccinated as many times as necessary. The vaccinated piglets are then exposed to C. coli approximately 3-20 days later, such as 4-10 days following the last immunization. Typically, vaccinated piglets are orally administered from 10⁶-10¹⁰, more particularly 10⁷-10⁹, such as 10⁸-10⁹ colony forming units (cfu) of C. coli, and indicia of Campylobacter infection are monitored, such as described in the examples herein. For example, the establishment of C. coli infection can be confirmed by examining tissue samples for bacteria and/or signs of inflammation, ulceration or carcinoma.

Alternatively, gnotobiotic piglets can first be infected with Campylobacter bacteria, such as C. coli bacteria in order to establish Campylobacter infection. For example, piglets can be orally inoculated at 1-5 days of age with C. coli, in an amount sufficient to cause infection, such as with 10⁶-10¹⁰, more particularly 10⁷-10⁹, such as 10⁸, cfu of C. coli. The presence of C. coli infection can be confirmed by examining tissue samples for bacteria and/or signs of inflammation, ulceration or carcinoma. In alternative embodiments, C. coli-inoculated pigs can be fed a milk-replacement diet that contains a dietary source of fermentable carbohydrate in order to cause gastroesophageal ulceration (GEU) of the pars esophagea. In this embodiment, pigs are typically fed a replacement formula well known in the art, such as but not limited to SIMILAC or ESBILAC, supplemented with a source of fermentable carbohydrate, such as but not limited to corn syrup, cornstarch, inulin, lactulose, wheat starch, sugar beet pulp, raffinose, stachyose, any of several oligosaccharides such as fructooligosaccharides, transgalactooligosaccharides, glucooligosaccharides, mannanoligosaccharides, xylooligosaccharides, or combinations of the above. Carbohydrate supplementation is typically introduced gradually, for example 2-7% (v/v), preferably 3-6% (v/v), such as 5% (v/v), beginning 1-10 days after C. coli inoculation, such as beginning at 3-8 days, preferably 2-4 days after C. coli inoculation. The amount of carbohydrate can be increased to, e.g., 8-15% (v/v), typically 9-12% (v/v), such as 10% (v/v), when the piglets accommodate to the additive, typically after 3-14 days following initial supplementation, generally 5-10 days following initial supplementation.

Once the bacterial infection has been established and, if desired, the milk-replacement diet described above is administered, a compound or a series of compounds can be delivered to the infected piglet at various times and in various dosages, depending on the particular goals of the screen. In a variation of this procedure, it may be desirable to administer the bacteria with a compound to determine whether, relative to control animals, the compound can effectively prevent in vivo the initial bacterial adhesion and/or the subsequent establishment of infection or pathogenesis.

Thus, the infected piglets can be used to screen for compounds and conditions which prevent Campylobacter infection, such as compounds and conditions that block binding of Campylobacter pathogens to the gut epithelium and/or that ameliorate the Campylobacter-associated pathogenesis of gastritis and small intestinal carcinoma, such as immunoproliferative small intestinal disease (IPSID). The efficacy of the compound or compounds can be assessed by examining at selected times the cells of the gut epithelial tissue of the infected animals for the presence or loss of Campylobacter bacteria and/or the development, inhibition, or amelioration of ulcer or tumor formation relative to appropriate control animals, for example, untreated C. coli-infected animals. The animal models described herein therefore provide the ability to readily assess the efficacy of various drugs or compounds based on different modes of administration and compound formation.

In addition to using the Campylobacter-infected animals to screen for therapeutic compounds, these animals can also be used to screen for conditions or stimuli which effect a block in or ameliorate Campylobacter, infection and/or associated gut diseases. Such stimuli or conditions include environmental or dietary changes, changing the gastrointestinal pH, or combinations of various stimuli or conditions which result in stress on the animal or on Campylobacter bacteria in the gut. Thus, for example, C. coli-infected animals can be exposed to a selected stimulus or condition, or a combination of stimuli or conditions, to be tested. The gut epithelial tissue of exposed animals is then examined periodically for a change in the number of Campylobacter bacteria and/or the disease state of the epithelial tissue relative to non-exposed control animals.

Another type of condition that can be tested for in the Campylobacter-inoculated animals described herein is the induction of an inflammatory response, for example by administering a chemical agent such as dextran sulfate, at various times prior to, during, or after administration of Campylobacter to the gnotobiotic piglet. The inflammatory agent can be administered orally or by any other mode that results in a gastrointestinal inflammatory response. The severity of the inflammatory response can be controlled by varying the dose and the duration of treatment with the chemical agent.

Campylobacter Vaccines

As explained above, the animal models described herein can be used to identify Campylobacter vaccines, such as C. coli vaccines, useful for treating and/or preventing Campylobacter infection in swine such as caused by C. coli. Campylobacter vaccines useful against C. coli and other Campylobacter infection can take various forms, such as inactivated or attenuated Campylobacter vaccines, as well as killed whole cell vaccines, such as formalin-killed Campylobacter vaccines, subunit vaccines and lysates.

A number of Campylobacter immunogens, will also find use in the subject vaccines. For example, vaccines that contain immunogens with one or more epitopes derived from any of the various Campylobacter flagellins, including but not limited to C. coli flagellins, such as FlaA and/or FlaB (Martin et al., Infect. Immun. (1989) 57:2542-2546; Wenman et al., J. Clin. Microbial. (1985) 21:108-112); any of the various Campylobacter cytotoxins such as the cytolethal distending toxin (CDT) (Pickett et al., Infect. Immun. (1996) 64:2070-2078), encoded by the cdt gene cluster, including cdtA, cdtB and cdtC; phospholipase A(2) (Istivan et al., J. Med. Microbial. (2004) 53:483-493); Campylobacter enterotoxins (Lindblom et al., J. Clin. Microbial. (1989) 27:1272-1276); adhesins, such as CadF (Konkel et al., J. Clin. Microbial. (1999) 37:510-517; Konkel et al., Malec. Microbial. (1997) 24:953-963); the lipoprotein encoded by ceuE (Park and Richardson, J. Bacterial. (1995) 177:2259-2264; Gonzalez et al., J. Clin. Microbial. (1997) 35:759-763); the plasmid pVir (Bacon et al., Infect. Immun. (2000) 68:4384-4390); PEB I (Pei and Blaser, J. Biol. Chem. (1991) 59:2259-2264; Pei et al., Infect. Immun. (1998) 66:938-943); pilin subunit proteins; Campylobacter glycolipids, such as the lipo-oligosaccharide (LOS) and/or capsular polysaccharide (CPS) (Karlyshev et al., in Campylobacter: molecular and cellular biology (2005) Eds. Ketley and Konkel, Chapter 12). See, e.g., Campylobacter, 2nd Edition (2000) Eds. Nachamkin and Blaser; Bang et al., J. Appl. Microbial. (2003) 94:1003-1014; Campylobacter: molecular and cellular biology (2005) Eds. Ketley and Konkel, for a description of these and other Campylobacter immunogens that can be present in such vaccines.

The immunogens for use in vaccine compositions can be produced using a variety of techniques. For example, the immunogens can be obtained directly from Campylobacter bacteria, commercially available from, e.g., the American Type Culture Collection (ATCC), 10801 University Boulevard, Manassas, Va.

The Campylobacter immunogens from the bacteria can also be provided in a lysate, obtained using methods well known in the art. Generally, such methods entail extracting proteins from Campylobacter bacteria using such techniques as sonication or ultrasonication; agitation; liquid or solid extrusion; heat treatment; freeze-thaw techniques; explosive decompression; osmotic shock; proteolytic digestion such as treatment with lytic enzymes including proteases such as pepsin, trypsin, neuraminidase and lysozyme; alkali treatment; pressure disintegration; the use of detergents and solvents such as bile salts, sodium dodecylsulphate, TRITON, NP40 and CHAPS; fractionation, and the like. The particular technique used to disrupt the cells is largely a matter of choice and will depend on the culture conditions and any pre-treatment used. Following disruption of the cells, cellular debris can be removed, generally by centrifugation and/or dialysis.

One particular technique for obtaining a Campylobacter lysate, such as a C. coli lysate vaccine composition uses proteolytic digestion, according to a method similar to the digestion protocol described in Waters et al. (2000) Vaccine 18:711-719. In this technique, C. coli bacteria are recovered by centrifugation and the bacterial pellet is resuspended, frozen and lyophilized. For bacterial digestion, pepsin is incubated with the lyophilized bacteria for 24-30 hours at 37 degrees C.

The immunogens present in such lysates can be further purified if desired, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

The Campylobacter immunogens can also be generated using recombinant methods, well known in the art. In this regard, oligonucleotide probes can be devised based on the sequence of the particular Campylobacter genome and used to probe genomic or cDNA libraries for Campylobacter genes encoding for the antigens useful in the present invention. The genes can then be further isolated using standard techniques and, if desired, restriction enzymes employed to mutate the gene at desired portions of the full-length sequence.

Similarly, Campylobacter genes can be isolated directly from bacterial cells using known techniques, such as phenol extraction, and the sequence can be further manipulated to produce any desired alterations. See, e.g., Sambrook et al., supra, for a description of techniques used to obtain and isolate DNA. Finally, the genes encoding the Campylobacter immunogens can be produced synthetically, based on the known sequences. The nucleotide sequence can be designed with the appropriate codons for the particular amino acid sequence desired. In general, one will select preferred codons for the intended host in which the sequence will be expressed. The complete sequence is generally assembled from overlapping oligonucleotides prepared by standard methods and assembled into a complete coding sequence. See, e.g., Edge, Nature (1981) 292:756; Nambair et al., Science (1984) 223:1299; Jay et al., J. Biol. Chem. (1984) 259:6311.

Once coding sequences for the desired polypeptides have been isolated or synthesized, they can be cloned into any suitable vector or replicon for expression in a variety of systems, including insect, mammalian, bacterial, viral and yeast expression systems, all well known in the art. In particular, host cells are transformed with expression vectors which include control sequences operably linked to the desired coding sequence. The control sequences will be compatible with the particular host cell used. It is often desirable that the polypeptides prepared using the above systems be fusion polypeptides. As with nonfusion proteins, these proteins may be expressed intracellularly or may be secreted from the cell into the growth medium.

Furthermore, plasmids can be constructed which include a chimeric gene sequence, encoding e.g., multiple Campylobacter antigens. The gene sequences can be present in a dicistronic gene configuration. Additional control elements can be situated between the various genes for efficient translation of RNA from the distal coding region. Alternatively, a chimeric transcription unit having a single open reading frame encoding the multiple antigens can also be constructed. Either a fusion can be made to allow for the synthesis of a chimeric protein or alternatively, protein processing signals can be engineered to provide cleavage by a protease such as a signal peptidase, thus allowing liberation of the two or more proteins derived from translation of the template RNA. The processing protease may also be expressed in this system either independently or as part of a chimera with the antigen and/or cytokine coding region(s). The protease itself can be both a processing enzyme and a vaccine antigen.

Depending on the expression system and host selected, the immunogens of the present invention are produced by growing host cells transformed by an expression vector under conditions whereby the immunogen of interest is expressed. The immunogen is then isolated from the host cells and purified. If the expression system provides for secretion of the immunogen, the immunogen can be purified directly from the media. If the immunogen is not secreted, it is isolated from cell lysates. The selection of the appropriate growth conditions and recovery methods are within the skill of the art.

The Campylobacter immunogens may also be produced by chemical synthesis such as by solid phase or solution peptide synthesis, using methods known to those skilled in the art. Chemical synthesis of peptides may be preferable if the antigen in question is relatively small. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag, Berlin (1984) and E. Gross and J. Meienhofer, Eds., The Peptides: Analysis, Synthesis, Biology, supra, Vol. 1, for classical solution synthesis.

The Campylobacter immunogens can be formulated into compositions, such as vaccine compositions, either alone or in combination with other antigens, for use in immunizing porcine subjects as described below. Methods of preparing such formulations are described in, e.g., Remington's Pharmaceutical Sciences, Mack Publishing Company, Easton, Pa., 18 Edition, 1990. The vaccines of the present invention can be prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Additional vaccine formulations include suppositories and, in some cases, aerosol, intranasal, oral formulations, and sustained release formulations. For sup-positories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and Hytrel copolymers, swellable polymers such as hydrogels, or resorbable polymers such as collagen and certain polyacids or polyesters such as those used to make resorbable sutures. The Campylobacter immunogens can also be delivered using implanted mini-pumps, well known in the art.

The Campylobacter immunogens can also be administered via a carrier virus which expresses the same. Carrier viruses which will find use with the instant invention include but are not limited to the vaccinia and other pox viruses, adenovirus, and herpes virus. By way of example, vaccinia virus recombinants expressing the novel proteins can be constructed as follows. The DNA encoding the particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the instant protein into the viral genome. The resulting TIC recombinant can be selected by culturing the cells in the presence of 5-bromodeoxyuridine and picking viral plaques resistant thereto.

Adjuvants which enhance the effectiveness of the vaccine may also be added to the formulation. Adjuvants may include for example, muramyl dipeptides, pyridine, aluminum hydroxide, alum, Freund's adjuvant, incomplete Freund's adjuvant (ICFA), dimethyldioctadecyl ammonium bromide (DDA), oils, oil-in-water emulsions, saponins, cytokines, and other substances known in the art. Bacterial toxins and bioadhesives are preferred adjuvants for use with mucosally-delivered vaccines, such as nasal vaccines. Such adjuvants include detoxified mutants of a bacterial ADP-ribosylating toxin such as a cholera toxin (CT), a pertussis toxin (PT), or an E. coli heat-labile toxin (LT). Adjuvants as described above are well known and commercially available from a number of sources, e.g., Difco, Pfizer Animal Health, Newport Laboratories, etc.

The immunogens may also be linked to a carrier in order to increase the immunogenicity thereof. Suitable carriers include large, slowly metabolized macro-molecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles.

The immunogens may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

Furthermore, the immunogens may be formulated into vaccine compositions in either neutral or salt forms. Pharmaceutically acceptable salts include the acid addition salts (formed with the free amino groups of the active polypeptides) and which are formed with inorganic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine formulations will contain a “therapeutically effective amount” of the active ingredient, that is, an amount capable of eliciting an immune response in a subject to which the composition is administered. In the treatment and prevention of Campylobacter infection in pigs, a “therapeutically effective amount” is readily determined by one skilled in the art using standard tests. The Campylobacter immunogens will typically range from about 1% to about 95% (w/w) of the composition, or even higher or lower if appropriate. With the present vaccine formulations, 0.1 to 500 mg of active ingredient per ml, preferably 1 to 100 mg/ml, more preferably 10 to 50 mg/ml, such as 20 . . . 25 . . . 30 . . . 35 . . . 40, etc., or any number within these stated ranges, of injected solution should be adequate to raise an immunological response when a dose of 0.25 to 3 ml per animal is administered. The quantity to be administered depends on the animal to be treated, the capacity of the animal's immune system to synthesize antibodies, and the degree of protection desired. Effective dosages can be readily established by one of ordinary skill in the art through routine trials establishing dose response curves.

To immunize a subject, the vaccine can be administered parenterally, such as by intramuscular injection, or via subcutaneous, intraperitoneal or intravenous injection. The subject is immunized by administration of the vaccine in at least one dose, and preferably two or more doses. Moreover, the animal may be administered as many doses as is required to maintain a state of immunity to infection. In one embodiment, a sow can be immunized parenterally to impart passive transfer to a fetus, and the newborn and young pigs can be subsequently vaccinated orally.

An alternative route of administration involves gene therapy or nucleic acid immunization. Thus, nucleotide sequences (and accompanying regulatory elements) encoding the Campylobacter immunogens can be administered directly to a subject for in vivo translation thereof. Alternatively, gene transfer can be accomplished by transfecting the subject's cells or tissues ex vivo and reintroducing the transformed material into the host. DNA can be directly introduced into the host organism, i.e., by injection (see International Publication No. WO/90/11092; and Wolff et al. (1990) Science 247:1465-1468). Liposome-mediated gene transfer can also be accomplished using known methods. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219 A; and Nabel et al. (1990) Science 1990) 249:1285-1288. Targeting agents, such as antibodies directed against surface antigens expressed on specific cell types, can be covalently conjugated to the liposomal surface so that the nucleic acid can be delivered to specific tissues and cells susceptible to infection.

The compositions of the present invention can be administered prior to, subsequent to or concurrently with traditional antimicrobial agents used to treat Campylobacter disease, such as but not limited to bismuth subsalicylate, metronidazole, amoxicillin, omeprazole, clarithromycin, ciprofloxacin, erythromycin, tetracycline, nitrofurantoin, ranitidine, omeprazole, and the like. One particularly preferred method of treatment is to first administer conventional antibiotics as described above followed by vaccination with the compositions of the present invention once the Campylobacter infection has cleared.

### 3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### Isolation of C. coli

Four isolates of Campylobacter species were isolated from each of 4 pigs (Group A) and 3 isolates from each of 3 pigs (Group B). Gram stain analysis revealed short negative rods with a ‘seagull’ morphology. Isolates were tested via biochemical analysis. Results are shown in Table 1.

TABLE 1

| | | | | | | | | Growth | | |
|---|---|---|---|---|---|---|---|---|---|---|
| | Sodium | | | | Growth | Growth | Growth | 10% | | |
| Pig isolate | hippurate | Catalase | Oxidase | Urease | at 25 C. | at 42 C. | O₂ + 37 C. | CO₂ | NA | Cph |
| 3-Group A | − | + | + | − | − | + | − | + | R | R |
| 4-Group A | − | + | + | − | − | + | − | + | S | S |
| 6-Group A | − | + | + | − | − | + | − | + | S | R |
| 7-Group A | − | + | + | − | − | + | − | + | S | R |
| 1-Group B | − | + | + | − | − | + | − | + | S | S |
| 2-Group B | − | + | + | − | − | + | − | + | S | R |
| 3-Group B | − | + | + | − | − | + | − | + | S | R |

NA = nalidixic acid;
Cph = cephalothin;
S = sensitive;
R = resistant

Based on biochemical analysis, isolates 6 (Group A), 7 (Group A), 2 (Group B) and 3 (Group B) were found to be Campylobacter coli, isolate 3 (Group A) was C. lari, and isolates 4 (Group A) and 1 (Group B) were C. upsaliensis. Due to the variability recognized with nalidixic acid and cephalothin antibiotic sensitivity and resistance testing, isolates were tested by the polymerase chain reaction (PCR) to confirm the species of Campylobacter isolated in each of the 7 pigs. Two previously published PCR protocols were used to evaluate the isolates:

1) Wang et al. Colony multiplex PCR assay for identification and differentiation of Campylobacter jejuni, C. coli, C. lari, C. upsaliensis, and C. fetus subsp. fetus. J Clin Microbiol 2002, p. 4744-47.

2) Klena et al. Differentiation of Campylobacter coli, C. jejuni, C. lari, and C. upsaliensis by a multiplex PCR developed from the nucleotide sequence of the lipid A gene lpxA. J Clin Microbiol 2004, p. 5549-57.

Both PCR protocols (Wang and Klena) found all 7 isolates to be C. coli based on the glyA gene (Wang) and the lpxA gene (Klena).

### Example 2

### Immunogenicity of Campylobacter coli Lysate Vaccine

In order to determine the infectivity and protective capability of C. coli, the following experiments were conducted.

A. Preparation of C. coli Lysates

C. coli lysates were prepared using proteolytic digestion, according to a method similar to the digestion protocol described in Waters et al. (2000) Vaccine 18:711-719. In particular, suspensions of C. coli bacteria propagated in liquid cultures of Brucella broth (Difco) supplemented with 10% fetal bovine serum (B-FBS) under microaerophilic conditions were allowed to reach approximately 10⁹ bacteria per ml. The bacteria were recovered by centrifugation (2000-30001×g) for 10 minutes. The spent supernatant was discarded and the bacterial pellet was resuspended in a minimal amount of Dulbecco's phosphate-buffered saline, transferred to a plastic cryo vial and frozen at −70 degrees C. While frozen, the bacterial pellet was lyophilized in a centrifugal evaporator apparatus (speed vac). Lyophilized bacterial pellets were pooled and weighed. For bacterial digestion, pepsin (Sigma, St. Louis, Mo.) at a concentration of 1.0 μg/ml was prepared by dilution into 10 mM HCl, pH 1.9-2.2. 1 μg of pepsin was incubated with 1 mg of lyophilized bacteria for 24-25 hours at 37 degrees C. on a magnetic stirrer. After completion of digestion, the digest was stored at −70 degrees C. until use.

B. Vaccination and Challenge Protocol

The lysates were formulated into vaccine compositions and used to vaccinate 10 conventional pigs as follows. The lysate was found to have a concentration of 3.05 mg/mL. The formulation administered to each pig transdermally was 25 μL of lysate+75 μL of phosphate-buffered saline 0.02 M (pH adjusted to 7.0)+100 μL of a commercial adjuvant (approved for use in swine). The vaccine was emulsified in adjuvant and the mixture was injected into the cervical region of each piglet. Each piglet received 1-200 μL injection transdermally at 7 and 35 days of age.

Vaccinated pigs were orally inoculated with C. coli organisms (roughly 10⁸ to 10⁹ colony forming units (cfu) in 2.0 ml of inoculum) 21 days after the last vaccination. On days 22, 23 and 24 pigs were offered 1.0 L of C. coli organisms (roughly 10⁸ to 10⁹ colony forming units (cfu) per mL) diluted with corn syrup in trays for consumption. All pigs were observed to feed on the offering. The pigs were terminated 28 days after challenge and efficacy of the vaccination was determined by a combination of gross and histologic examination of the stomachs and by Enzyme linked immunosorbent assay (ELISA).

C. C. coli ELISA

A C. coli ELISA was developed and performed as follows. C. coli antigen was prepared by harvest of 500 mL of actively growing culture (10⁸ to 10⁹ organisms per mL) in B-FBS. The bacteria were recovered by centrifugation (2000-3000×g) for 10 minutes. The spent supernatant was discarded and the bacterial pellet was resuspended in 2 mL of Dulbecco's phosphate-buffered saline. This wash was repeated. The 2 mL volume of resuspended bacteria was sonicated (on ice) at 20 kHz, 50% duty cycle, amplitude of 4 for 60 seconds. The sonicate was then centrifuged at 3000 g for 10 minutes and supernatant filter sterilized (0.450 and protein concentration determined.

C. coli sonicate=1.17 mg/mL used at 1:8000 (1.54 in 12 mL buffer)

Final concentration of Ag=0.15 μg/mL or 0.015 μg per well for each Ag)

PBST: 10×PBS diluted 1:10 with double distilled water; 0.05% TWEEN 20

Block: Carbonate coating buffer+0.2% gelatin (Bovine Gelatin)

Solution for dilution of Antibody: PBST+0.2% gelatin (Bovine gelatin)

0.1 g of gelatin added to 50 mL of solution and dissolved in a 57 degree C. water bath for 20 minutes, and cooled.

A 96-well plate was coated with 100 μL of coli Ag per well at a concentration of 0.15 μg/mL. The diluent used for coating the plates was 1× Carbonate Coating Buffer. 12 mL of Ag preparation was made per plate; therefore, 1.5 μL of coli Ag was present per 12 mL volume. Plates were covered with adhesive seal and cover to keep light out and left at room temperature for 24 hours or overnight. Plates were washed 4 times with PBST. 100 μL of block was added and plates were incubated for 30 minutes at 37 degrees C. (or 1 hour at room temperature). Plates were washed 2 times with PBST.

Primary antibody (pig sera) was diluted 1:800 in PBST+0.2% gelatin. 100 μL per well were added and plates were incubated for 1 hour at 37 degrees C. (or 2 hours at room temperature). Plates were washed 4 times with PBST. Secondary antibody was diluted (protein A-HRP conjugate at a concentration of 1:5000) in PBST+0.2% gelatin. 100 μL per well was added and incubated for 1 hour at 37 degrees C. (or 2 hours at room temperature). Plates were washed 4 times with PBST.

100 μL of tetramethylbenzidine substrate (TMB) was added per well and incubated at room temperature until color developed. 50 μL of TMB stop solution (1M sulfuric acid) was added per well. Plates were read at 450λ. Results of the C. coli ELISA on the plasma of each of the 10 vaccinated and challenged pigs are shown in Table 2.

TABLE 2

| Pig ID | Vx 1 | Vx 2 | Challenge | Necropsy |
|---|---|---|---|---|
| W01 | 31 | 84 | 100 | 100 |
| W08 | 41 | 0 | 100 | 100 |
| W11 | 5 | 100 | 100 | 100 |
| W15 | 10 | 100 | 100 | 100 |
| W16 | 0 | 3 | 100 | 100 |
| W17 | 42 | 16 | 100 | 100 |
| W20 | 21 | 92 | 100 | 100 |
| W23 | 24 | 69 | 100 | 100 |
| W25 | 5 | 100 | 100 | 100 |
| W27 | 1 | 19 | 100 | 100 |

D. Indices of Efficacy

The in vivo safety of the vaccine preparations was determined in uninfected conventionally-reared swine. For this, vaccine digests were administered as above and clinical evidence for endotoxin-mediated damage (diarrhea, hypovolemic shock, respiratory distress and sudden death) were monitored. All preparations were judged as safe by this process.

The efficacy of the vaccine preparations in inducing humoral responses in vaccinates was determined by an ELISA assay for IgG and/or IgM antibodies to undigested C. coli cell lysates in post-vaccinal sera. The vaccine digest was considered to be effective since it induced high concentrations of specific antibody. Further documentation of clinical efficacy is whether and/or reduced recoverable bacteria from gastric homogenates or reduced bacterial cfu an average of 2 logs below the challenge control pigs, as determined by Q-PCR. Additional parameters of success include reduced or absent gastric inflammation (gastritis) and the absence of either gastric mucosal ulcers or ulcers of the pars esophagea.

### Example 3

### Production of an Animal Model of Porcine GEU

In order to produce an animal model for porcine GEU the following experiment is conducted.

### Materials and Methods

A. Gnotobiotic Piglets

A total of 22 gnotobiotio piglets from portions of 4 litters are used in these experiments. These are derived by Caesarian section and raised as described elsewhere (Krakowka and Eaton “Helicobacter pylori infection in gnotobiotic piglets: A model of human gastric bacterial disease” in Advances in Swine in Biomedical Research II, Tumbleson et al., eds., Plenum Press, New York, N.Y., 779-810). The basic diet for these piglets consists of a sterile liquid sow milk replacement formula (SIMILAC) individually fed to each piglet in feed pans three times daily, 200-300 ml/feeding. The volume of diet is adjusted over time to accommodate the increased nutritional requirements of the growing piglets. Dietary supplementation with liquid carbohydrate is accomplished by adding sterile corn syrup (KARO) at 5% (v/v) at 5-7 days of age (Krakowka et al. (1998) Vet. Pathol. 35:274-282). This is increased to 10% (v/v) at 10-12 days of age and continued to termination at days 30-35 of age or when moribund

B. Bacterial Inocula

C. coli: A total of 11 piglets (Group B), separately housed from Group A pigs above and the controls (Group C), are inoculated with porcine C. coli (10⁸ bacterial cfu contained in 2.0 ml Brucella broth) at 3 days of age, obtained as described above. Two piglets of Group C receive Brucella broth alone as uninfected controls.

C. Experimental Design and Pathologic Evaluation

The basic design used in this study is similar to that described in Krakowka et al. (1998) Vet. Pathol. 35:274-282. Separately housed piglet groups are fasted for 12 hrs and orally inoculated with bacteria at 3 days of age. Supplementation with carbohydrate into the diet is introduced gradually (5%) starting at 5 to 7 days of age and increased to 10% (v/v) when it appears that the piglets have accommodated to this additive, usually by 10-14 days of age. Three Campylobacter-infected piglets receive carbohydrate supplementation. At planned termination at 30-35 days of age (post-infection days 27, 32) piglets are fasted overnight, sedated and removed from the isolation units. After collection of a terminal clotted blood sample for serum, piglets are euthanatized with an intravenous overdose of sodium pentothal (EUTHOL). The stomachs are isolated, ligated at the distal esophagus and proximal duodenum and removed. Using sterile methods, the stomach is opened along the greater and lesser curvatures.

When culture and re-isolation is performed, one-half of the stomach is used for microbiology as described in Krakowka and Eaton “Helicobacter pylori infection in gnotobiotic piglets: A model of human gastric bacterial disease” in Advances in Swine in Biomedical Research II, Tumbleson et al., eds., Plenum Press, New York, N.Y., 779-810; Krakowka et al. (1998) Vet. Pathol. 35:274-282; and PCT Publication No. WO 2004/069184. For this, the mucosa is scraped free of the muscularis, weighed and then homogenized in Brucella broth as a 10% (w/v) suspension. Ten-fold dilutions of gastric homogenate are then plated in duplicate onto Skirrow's medium plates and these are incubated for 4 days, 37 C, 5% (v/v) oxygen. Re-isolates are confirmed to be Campylobacter species as described in Example 1.

For pathologic evaluation, all mucosal ulcers and GEU are photographed as fresh specimens before emersion fixation of the opened stomachs in 10% (v/v) phosphate-buffered formalin solution for 24 hrs. Representative sections of the nonglandular (esophageal) cardia, the glandular cardia, the fundus, the antrum, pylorus and proximal duodenum are collected, and processed into paraffin blocks by routine methods. Replicate 5 micron tissue sections are de-paraffinized through graded alcohols, rehydrated and stained with hematoxylin and eosin and Warthin Starry silver stains as described in Krakowka and Eaton “Helicobacter pylori infection in gnotobiotic piglets: A model of human gastric bacterial disease” in Advances in Swine in Biomedical Research II, Tumbleson et al., eds., Plenum Press, New York, N.Y., 779-810 and Krakowka et al. (1998) Vet. Pathol. 35:274-282.

Thus, animal models for the study of Campylobacter infection, as well as methods for preventing Campylobacter infection are described, as well as compositions for use with the methods. Although preferred embodiments of the subject invention have been described in some detail, it is to be understood that obvious variations can be made without departing from the spirit and scope of the invention as defined by the claims.

1. A method of treating or preventing Campylobacter infection in a porcine subject comprising administering to said subject a therapeutically effective amount of a composition comprising at least one Campylobacter immunogen.

2. The method of claim 1, wherein the composition comprises at least one C. coli immunogen.

3. The method of claim 2, wherein the composition comprises a C. coli lysate.

4. The method of claim 3, wherein the lysate is produced by proteolytic digestion of C. coli bacteria.

5. The method of 1, wherein the composition further comprises an adjuvant.

6. The method of 1, wherein the composition is administered orally.

7. A method for infecting a gnotobiotic piglet with a porcine isolate of Campylobacter, said method comprising:
(a) isolating Campylobacter from a porcine subject; and
(b) administering a dose of the Campylobacter isolate to said gnotobiotic piglet in an amount sufficient to cause Campylobacter infection.

8. The method of claim 7, wherein the Campylobacter isolate is C. coli.

9. The method of claim 7, of wherein the Campylobacter isolate is administered orally to the piglet.

10. The method of claim 9, wherein the Campylobacter is administered in an amount of 10⁷-10⁹ colony forming units.

11. A method for evaluating the ability of a vaccine to prevent Campylobacter infection comprising:
(a) administering to a porcine subject a candidate vaccine;
(b) exposing the porcine subject from step (a) to a Campylobacter isolate in an amount sufficient to cause infection in an unvaccinated subject; and
(c) observing the incidence of Campylobacter infection in the porcine subject, thereby evaluating the ability of the candidate vaccine to prevent Campylobacter infection.

12. The method of claim 11, wherein the candidate vaccine is a C. coli vaccine comprising at least one C. coli immunogen and the Campylobacter isolate is a C. coli isolate.

13. The method of claim 11, wherein the porcine subject is a gnotobiotic piglet.

14. A method of producing a porcine animal model of gastroesophageal ulceration (GEU) of the pars esophagea, said method comprising:
(a) isolating Campylobacter from a porcine subject;
(b) exposing a gnotobiotic piglet to the Campylobacter isolate in an amount sufficient to cause infection in said piglet; and
(c) feeding said infected piglet a milk-replacement diet that contains a dietary source of fermentable carbohydrate under conditions sufficient for producing GEU of the pars esophagea.

15. The method of claim 14, wherein the Campylobacter isolate is a C. coli isolate.

16. The method of claim 14, wherein the Campylobacter isolate is administered orally to the piglet.

17. The method of claim 16, wherein the Campylobacter isolate is administered in an amount of 10⁷-10⁹ colony forming units.

18. The method of claim 14, wherein said dietary source of fermentable carbohydrate is corn syrup.

19. A method of producing a porcine animal model of gastroesophageal ulceration (GEU) of the pars esophagea, said method comprising:
(a) isolating C. coli from a porcine subject;
(b) orally administering 10⁷-10⁹ colony forming units of the C. coli isolate to a gnotobiotic piglet in order to cause C. coli infection; and
(c) feeding said infected piglet a milk-replacement diet that contains corn syrup as a fermentable source of carbohydrate under conditions sufficient for producing GEU of the pars esophagea.

20. A method of identifying a compound capable of treating Campylobacter infection, said method comprising:
(a) exposing a gnotobiotic piglet to a Campylobacter isolate in an amount sufficient to cause infection in said piglet;
(b) delivering a compound or series of compounds to said infected piglet; and
(c) examining the piglet from step (b) for the presence or loss of Campylobacter bacteria and/or the development, inhibition, or amelioration of ulcer or tumor formation relative to an untreated Campylobacter-infected gnotobiotic piglet.

21. A method of identifying a compound capable of treating C. coli infection, said method comprising:
(a) providing a porcine animal model of GEU produced by the method of claim 14;
(b) delivering a compound or series of compounds to said infected piglet; and
(c) examining the piglet from step (b) for the presence or loss of C. coli bacteria and/or the development, inhibition, or amelioration of ulcer or tumor formation relative to an untreated C. coli-infected gnotobiotic piglet.

22. (canceled)

23. (canceled)

24. A method for preventing food-borne transmission of Campylobacter pathogens to humans due to consumption of pork, said method comprising administering to a porcine subject a therapeutically effective amount of a composition comprising at least one Campylobacter immunogen.

25. The method of claim 24, wherein the composition comprises at least one C. coli immunogen.

26. The method of claim 25, wherein the composition comprises a C. coli lysate.

27. The method of claim 26, wherein the lysate is produced by proteolytic digestion of C. coli bacteria.

28. The method of claim 24, wherein the composition further comprises an adjuvant.

29. The method of claim 24, wherein the composition is administered orally.
